(19) United States
(12) Patent Application Publication
(10) Pub. No.: US 2018/0015007 A1
(43) Pub. Date: Jan. 18, 2018

(54) POLYMERIC MICELLE CARRIER COMPOSITION AND POLYMERIC MICELLE COMPOSITION

(71) Applicant: NanoCarrier Co., Ltd., Kashiwa-shi (JP)

(72) Inventors: Kenta ISHII, Kashiwa-shi (JP); Yasuki KATO, Kashiwa-shi (JP)

(73) Assignee: NanoCarrier Co., Ltd., Kashiwa-shi (JP)

(21) Appl. No.: 15/553,309

(22) PCT Filed: Feb. 29, 2016
(86) PCT No.: PCT/JP2016/056101

§ 371 Date: Aug. 24, 2017

(30) Foreign Application Priority Data
Feb. 27, 2015 (JP) 2015-039241

(51) Int. Cl. A61K 8/02; A61K 47/42; A61K 47/34; A61K 9/107; A61K 8/92; A61K 47/44; A61K 8/06

(52) U.S. Cl. CPC A61K 8/0291; A61K 47/44; A61K 47/42; A61K 8/06; A61K 9/107; A61K 8/92; A61K 47/34

(57) ABSTRACT

A polymeric micelle carrier composition contains i) a block copolymer having a hydrophilic polymer chain segment and a hydrophobic polymer chain segment; ii) a charged surfactant; and iii) a fatty oil. The carrier composition can be utilized as a base material of a cosmetic composition or a pharmaceutical composition and excels in loadability of non-lipophilic drugs. One example of a non-lipophilic drug is a hair growth promoting peptide.

### TECHNICAL FIELD

The present invention relates to a polymeric micelle carrier composition that is applicable as a carrier of a cosmetic composition, and to a polymeric micelle composition in which a drug is loaded by the carrier composition.

### BACKGROUND ART

Block copolymers having a hydrophilic segment derived from poly(ethylene glycol) and a hydrophobic segment derived from poly(amino acid) form a polymeric micelle structure having a hydrophobic region in the inner shell portion caused by hydrophobic interactions between the polymers. Polymeric micelle technologies using such block copolymers, which utilize a micelle forming mechanism caused by the hydrophobic interactions, have been studied as a technique for encapsulating poorly water-soluble drugs, such as paclitaxel, which is a poorly water-soluble anticancer agent, in micelles without chemical bonding to the block copolymer (Patent Literature 1 or 2). Polymeric micelle technology has also been applied to a cosmetic composition containing hinokitiol, which is a poorly water-soluble drug and a skin whitening compound (Patent Literature 3).

On the other hand, it has been considered to be difficult to provide polymeric micelle compositions that excel in loadability of non-lipophilic drugs from a drug inclusion principle based upon a polymeric micelle technology that uses such hydrophobic interactions.

### CITATION LIST

### Patent Literature

Patent Literature 1: Japanese Patent No. 2777530

Patent Literature 2: WO 2004/082718

Patent Literature 3: WO 2008/026776

### SUMMARY OF THE INVENTION

### Problem(s) to be Solved by the Invention

One object of the present invention is to provide a polymeric micelle carrier composition capable of greatly improving the loadability of non-lipophilic drugs. Another object of the present invention is also to provide a polymeric micelle composition that excels in the loadability of non-lipophilic drugs.

### Means for Solving the Problem(s)

The present inventors found that, if a fatty oil, which has been considered to have, in principle, a poor affinity to non-lipophilic drugs, is intentionally used as one of the components of a polymeric micelle carrier composition, it contributes to a significant improvement of the loadability of the non-lipophilic drug by the carrier composition, and the present invention was completed.

The present invention provides a polymeric micelle carrier composition that is applicable as a carrier of a cosmetic composition, the polymeric micelle carrier composition comprising i) a block copolymer having a hydrophilic polymer chain segment and a hydrophobic polymer chain segment; ii) a charged surfactant; and iii) a fatty oil. In another aspect, the present invention also provides a polymeric micelle composition comprising the carrier composition, and a non-lipophilic drug loaded by the carrier composition.

### Effects of the Invention

According to the present invention, the loadability of a non-lipophilic drug in a polymeric micelle composition can be improved.

### MODES FOR CARRYING OUT THE INVENTION

The polymeric micelle carrier composition of the present invention contains a block copolymer, a charged surfactant, and a fatty oil. Although fatty oils have been considered to have, in principle, a poor affinity to non-lipophilic drugs, when a fatty oil is intentionally used as one of the components of the carrier composition in combination with the block copolymer and the charged surfactant, it contributes to a significant improvement of the loadability of the non-lipophilic drug by the carrier composition.

The fatty oil may be an oil composition selected from known vegetable, animal, and synthetic oils. More specifically, the fatty oil may be an oil composition that is selected from oils (fats), which are obtained from animals and vegetables and are liquid at 20° C. and at standard atmospheric pressure (101.325 kPa). Examples of the vegetable oil include olive oil, sesame oil, soybean oil, camellia oil, corn oil, rapeseed oil, castor oil, palm oil, peanut oil, cottonseed oil, avocado oil, sunflower oil, and almond oil. Examples of the animal oil include liver oil, fish oil, turtle oil, mink oil, and egg yolk oil. As used herein, the animal and vegetable oils include processed fatty oils obtained by hydrogenating the oils exemplified above.

As used herein, the term “non-lipophilic drug” refers to a drug having a maximum solubility in liquid paraffin at 20° C. and at standard atmospheric pressure (101.325 kPa) that is 100 mg/L or less, more strictly 10 mg/L or less.

The polymeric micelle composition of the present invention can be applied to a polymer compound as the non-lipophilic drug, more specifically to a biopolymer compound. Examples of the biopolymer compound include peptides, proteins (e.g., cytokines and antibodies), polysaccharides, glycoproteins, and nucleic acids (e.g., decoy oligonucleotides, antisense oligonucleotides, and siRNAs). The non-lipophilic drug is preferably in a charged (cationic or anionic) state. As used herein, the term “cationic” refers to a state in which the number of positive charges is greater than that of negative charges in an aqueous medium having a physiological pH (e.g., pH 7.4), and the term “anionic” refers to a state in which the number of negative charges is greater than that of positive charges in said aqueous medium.

The non-lipophilic drug may be a known low-molecular-weight or polymer compound used as a hair growth promoter, a skin whitener, an anti-inflammatory agent, an immunosuppressant, an antibacterial agent, an antifungal agent, an antibiotic, an antiviral agent, an antihistamine, an anticancer agent, or an anesthetic. Thus, the micelle composition may be in a state that it contains a hair growth promoter as the non-lipophilic drug. The hair growth promoter is preferably a drug exhibiting a new-hair growing effect, a hair-growth promoting effect, or a hair nourishing effect; more specifically, examples include finasteride, minoxidil, carpronium chloride, and known hair-growth promoting peptides. Examples of the skin whitener include azelaic acid, hydroquinone, and vitamin C and derivatives thereof (e.g., ascorbic acid, ascorbyl glucoside, ascorbyl phosphate salts, ascorbyl palmitate, ascorbyl tetrahexyldecanoate, arbutin, and ellagic acid). Examples of the anti-inflammatory agent include lidocaine, indomethacin, fentanyl, and ketoprofen. Examples of the immunosuppressant include tacrolimus hydrate and cyclosporine. Examples of the antifungal agent include oxiconazole nitrate, liranaftate, bifonazole, amorolfine hydrochloride, and clotrimazole. Examples of the antihistamine include fexofenadine hydrochloride, loratadine, azelastine hydrochloride, and oxatomide. Examples of the anticancer agent include 5-FU (5-fluorouracil) and bleomycin sulfate.

The charged surfactant may be a known surfactant that dissociates into ions in an aqueous solution and has a cationic or anionic surface active moiety. Examples of the cationic surfactant include cetylpyridinium chloride, dimethyldistearylammonium chloride, benzethonium chloride, and benzalkonium chloride. Examples of the anionic surfactant include sodium dodecylbenzenesulfonate, sodium octanoate, sodium lauryl phosphate, and sodium lauryl sulfate. If the non-lipophilic drug is in a charged state, a surfactant having a charge opposite to that of the drug is preferably used. More specifically, an anionic surfactant is preferably used for a cationic drug, and a cationic surfactant is preferably used for an anionic drug. Thus, the non-lipophilic drug preferably has a charge opposite to that of the charged surfactant. In addition, the micelle composition may be in a state containing a charged peptide as the non-lipophilic drug.

According to the present invention, the loading percentage of the non-lipophilic drug in the polymeric micelle composition can be greatly increased. More specifically, and as shown in Examples below, polymeric micelle compositions are provided, in which the loading percentage of the non-lipophilic drug exceeds, e.g., 20 mass % or more, or e.g., 30 mass % or more, or e.g., 40 mass % or more, or e.g., 70 mass % or more. It is noted that the term “loading percentage” in the present specification means a value calculated as follows: a non-lipophilic drug dissolved in 15 mL of a 100 mM phosphate buffer in a proportion of 5 parts by mass is mixed and stirred into a polymeric micelle carrier composition having 100 parts by mass in terms of the block copolymer; after standing still at 5° C. overnight the free amount of the non-lipophilic drug in the aqueous phase is measured by using high-performance liquid chromatography and is compared to the amount of the drug that was added to the carrier composition.

Although the reason why the loadability of the non-lipophilic drug can be improved according to the present invention is not certain, it is conjectured as follows. First, the polymeric micelle carrier composition of the present invention is radially arranged, in principle, as a structure in which the block copolymers have been oriented with the hydrophobic polymer chain segments inward and with the hydrophilic polymer chain segments outward, thereby surrounding the fatty oil; furthermore, the charged surfactant molecules are arranged around the fatty oil such that lipophilic moieties are oriented inward and hydrophilic moieties are oriented outward with the lipophilic moieties being attracted to the fatty oil. In addition, the polymeric micelle composition of the present invention is in the state in which, in principle, the non-lipophilic drug is attracted to and retained by the hydrophilic moieties of the charged surfactant as its structure. Thus, it is believed that the loading properties of the non-lipophilic drug in the micelle composition are improved by the charged surfactant serving as an anchor for holding the fatty oil in the state in which the non-lipophilic drug is captured, and by the fatty oil functioning as an anchor base for retaining the non-lipophilic drug in the micelle composition via the charged surfactant. As used herein, the expression “the loading of the non-lipophilic drug in the micelle composition” therefore is not limited to the state in which it is disposed in the hydrophobic region within the polymeric micelle composition formed by hydrophobic polymer chain segments of the block copolymers, but also the state in which it is disposed outside of the hydrophobic region (in the hydrophilic region formed by the hydrophilic polymer chain segments of the block copolymers).

In the block copolymer, the hydrophilic polymer chain segment may be a segment derived from poly(ethylene glycol), and the hydrophobic polymer chain segment may be a segment derived from poly(amino acid). The pair of terminal ends of the main chains of the hydrophilic polymer chain segment and the hydrophobic polymer chain segment may be bound by a covalent bond.

The number of repeating units of the hydrophilic polymer chain segment can be set to, e.g., 20 or more, or e.g., 45 or more, and can be set to, e.g., 1,000 or less, or e.g., 700 or less, or e.g., 450 or less. The molecular mass of the hydrophilic polymer chain segment can be set to, e.g., 1,000 Da or more, or e.g., 2,000 Da or more, or e.g., 5,000 Da or more, and can be set to, e.g., 40,000 Da or less, or e.g., 30,000 Da or less, or e.g., 20,000 Da or less.

The number of repeating units of the hydrophobic polymer chain segment can be set to, e.g., 10 or more, or e.g., 20 or more, and can be set to, e.g., 200 or less, or e.g., 100 or less, or e.g., 60 or less. The molecular mass of the hydrophobic polymer chain segment can be set to, e.g., 1,000 Da or more, or e.g., 2,000 Da or more, and can be set to, e.g., 30,000 Da or less, or e.g., 16,000 Da or less, or e.g., 10,000 Da or less.

The hydrophobic polymer chain segment of the block copolymer may be in a state having, for example, alkyl group side chain amino acids or aralkyl group side chain amino acids in a repeating unit. Examples of the alkyl group side chain amino acid include alanine, valine, leucine, and isoleucine. An example of the aralkyl group side chain amino acid includes phenylalanine. If it contains two or more residues of alkyl group side chain amino acids and/or aralkyl group side chain amino acids, they may be the same amino acid residues, or residues of two or more different types of alkyl group side chain amino acids and/or aralkyl group side chain amino acids may be mixed. The proportion of the residues of the alkyl group side chain amino acids or the aralkyl group side chain amino acids with respect to all of the repeating units of the hydrophobic polymer chain segment is not limited and may be, e.g., 20% or more, or e.g., 35% or more, or e.g., 40% or more, or e.g., 50% or more, or e.g., 80% or more, or e.g., 95% or more, or e.g., 99% or more, or e.g., 100%.

The molecular mass of the hydrophobic polymer chain segment with respect to the molecular mass 100% of the hydrophilic polymer chain segment can be set to, e.g., 10% or more, or e.g., 20% or more, and be set to, e.g., 400% or less, or e.g., 300% or less.

As examples of the structural formula of the block copolymer, Formulae (I) and (II) are mentioned:

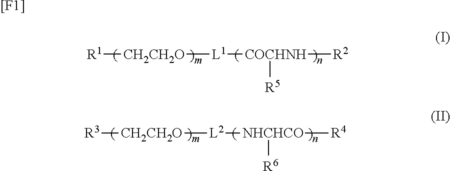

In Formulae (I) and (II), R¹ and R³ are each independently a hydrogen atom or a C₁₋₆ alkoxy, acryloxy, aryl C₁₋₃ oxy, cyano, carboxyl, amino, C₁₋₆ alkoxycarbonyl, C₂₋₇ acylamido, tri-C₁₋₆ alkylsiloxy, siloxy, or silylamino group; R² is a hydrogen atom, a saturated or unsaturated C₁-C₂₉ aliphatic carbonyl group, or an aryl carbonyl group; and R⁴ is a hydroxyl group, a saturated or unsaturated C₁-C₃₀ aliphatic oxy group, or an aryl-lower-alkyloxy group.

In Formulae (I) and (II), R⁵ and R⁶ are each independently a side chain of an amino acid. However, 50% or more, or e.g., 80% or more, or e.g., 95% or more, or e.g., 99% or more, or e.g., 100% of the n number of repeating units are a C₁-C₈ alkyl-amino acid or an aralkyl group side chain amino acid. Amino acid side chains from among R⁵ or R⁶, which are not a C₁-C₈ alkyl side chain or aralkyl side chain, may be a hydrophilic moiety having an OH group or a COOH group.

In Formulae (I) and (II), m is an integer of, e.g., 20 or more, or e.g., 45 or more, or is an integer of, e.g., 700 or less, or e.g., 450 or less. n is an integer of, e.g., 10 or more, or e.g. 20 or more, or is an integer of, e.g., 200 or less, or e.g., 100 or less, or e.g., 60 or less.

In Formulae (I) and (II), L¹ is a linking group selected from —NH—, —Z—NH—, —Z—, and —Z—S—Z—NH— (where Z is independently a C₁-C₆ alkylene group); and L² is a linking group selected from —Z—, —CO—Z—CO—, —Z—CO—Z—CO—, —NH—CO—Z—CO—, and —Z—NH—CO—Z—CO— (where Z is independently a C₁-C₆ alkylene group).

As other examples of the structural formula of the block copolymer, Formulae (III) and (IV) are mentioned:

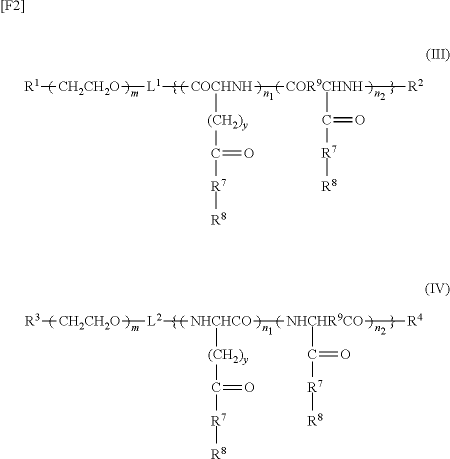

In Formulae (III) and (IV), the definitions of R⁴, R², R³, R⁴, m, L¹, and L² are the same as the definitions in Formulae (I) and (II).

In Formulae (III) and (IV), R⁷ is —O— or —NH—; R⁸ is a hydrogen atom, a phenyl, benzyl, —(CH₂)₄-phenyl, or unsubstituted or amino- or carbonyl-substituted C₄-C₁₆ alkyl group, or a residue of a sterol derivative; and R⁹ is a methylene group.

In Formulae (III) and (IV), n1 is an integer of 10 to 200; n2 is an integer of 0 to 200 (however, if n2 is 1 or more, the (COCHNH) units and the (COR⁹CHNH) unit(s) are present randomly; if n2 is 2 or more, R⁸s are independently and randomly selected in each amino acid unit in the block copolymer; and hydrogen atoms account for 75% or less of all the R⁸s); and y is 1 or 2.

As other examples of the structural formula of the block copolymer, Formulae (V) and (VI) are mentioned:

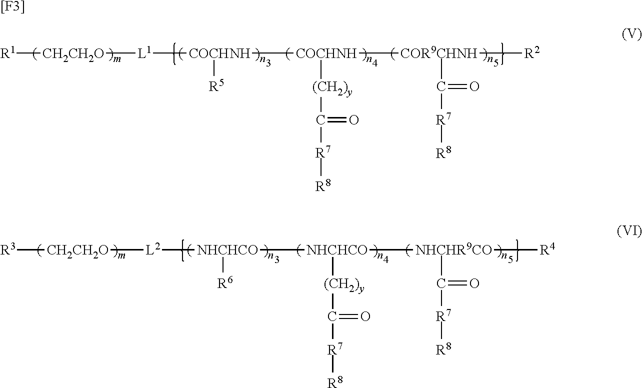

In Formulae (V) and (VI), the definitions of R⁴, R², R³, R⁴, R⁵, R⁶, L¹, and L² are the same as the definitions in Formulae (I) and (II), and the definitions of R⁷, R⁸, R⁹, and y are the same as the definitions in Formulae (III) and (IV).

In Formulae (V) and (VI), n3 is an integer of 1 to 200; n4 is an integer of 1 to 200; and n5 is an integer of 0 to 200. However, the n4 unit(s) and the n5 unit(s) (if n5 is 1 or more) are present randomly. The n3 unit(s), the n4 unit(s), and the n5 unit(s) (if n5 is 1 or more) may be present randomly, or may be present divided into a block composed of the n3 unit(s) and a block composed of the n4 unit(s) and the n5 unit(s) (if n5 is 1 or more). In addition, 50% or more, e.g., 80% or more, or e.g., 90% or more, or e.g., 95% or more, or e.g., 99% or more, or e.g., 100% from among the n3 repeating units are C₁-C₈ alkyl group side chain amino acids or aralkyl group side chain amino acids. Amino acid side chains, which are not a C₁-C₈ alkyl side chain or an aralkyl side chain, from among the n3 repeating units, may be a hydrophilic moiety having an OH group or a COOH group. In addition, the percentage of the number of the n3 unit(s) with respect to the total number of the n3 unit(s), the n4 unit(s), and the n5 unit(s) (if n5 is 1 or more) may be, e.g., 20% or more, or e.g., 35% or more, or e.g., 40% or more, or e.g., 50% or more, or e.g., 80% or more, or e.g., 90% or more.

The block copolymer can be formed by coupling, according to a known method, e.g., a polymer having a hydrophilic polymer chain and a polymer having a poly(amino acid) chain, either as is, or after purifying to narrow the molecular mass distribution if necessary. The block copolymer of Formula (I) can be prepared by, for example, forming a poly(ethylene glycol) chain by performing anionic living polymerization using an initiator capable of providing R¹, introducing an amino group at the propagating terminal end of the polymer chain, and polymerizing the desired amino acid, which contains an alkyl side chains, from the amino group terminal.

The mass percentage of the fatty oil to the block copolymer in the carrier composition or in the micelle composition may be, e.g., 50 mass % or less or e.g., 20 mass % or less. Although the mass percentage of the charged surfactant to the fatty oil in the carrier composition or the micelle composition may be 100 mass % or less, the content of the charged surfactant in the micelle composition is preferably adjusted such that it has a number of charges that is equal to or greater than the number of opposite charges that the non-lipophilic drug has.

The carrier composition can be formed, for example, according to the following. i) a block copolymer, a charged surfactant, and a fatty oil are added to an organic solvent to prepare a stock solution; ii) the organic solvent is removed from the stock solution; iii) the residue after the removal (e.g., a solid or paste) is added to water to prepare a suspension containing the block copolymer, the charged surfactant, and the fatty oil; and iv) the mixture of the block copolymer, the charged surfactant, and the fatty oil is dispersed in the suspension. The micelle composition can be formed by mixing a non-lipophilic drug with the carrier composition following the formation of the carrier composition, or with a previously-prepared carrier composition. The non-lipophilic drug may be mixed with the carrier composition in state of a drug solution containing the drug, or it may be mixed by adding it to a solution containing the carrier composition (e.g., the dispersion obtained in iv) above). Examples of the organic solvent include acetone, dichloromethane, dimethylformamide, dimethyl sulfoxide, acetonitrile, tetrahydrofuran, and methanol. The stock solution may contain two or more organic solvents and may also contain a small amount of water. The organic solvent(s) may be removed from the stock solution by a known technique, such as evaporation, extraction, or membrane separation. The water, in which the residue obtained after removal of the organic solvent(s) is added, may contain an additive, such as a salt or a stabilizer. With regard to the dispersion of the mixture, known micronizing means may be used, such as a sonicator, a high-pressure emulsifying machine, or an extruder.

The polymeric micelle carrier composition of the present invention is applicable as a carrier of a cosmetic composition or also as a carrier of a pharmaceutical composition. In addition, the polymeric micelle composition of the present invention can be used as a cosmetic composition as well as a pharmaceutical composition. It is noted that, in the present specification, quasi drugs are considered to be included within cosmetics. Because the polymeric micelle composition of the present invention can use its characteristic properties and permeate and stably remain in skin tissue (within the epidermal layer) from within the epidermis to the outside of the dermis, it is suitable for use as a topical skin preparation. For example, when a micelle composition containing a hair growth promoter as the non-lipophilic drug is administered to the skin, the micelle composition stays around the hair roots and the hair growth promoter can be released in the vicinity of hair roots in a sustained manner. In this way, the polymeric micelle composition of the present invention can be used as a cosmetic composition or as a pharmaceutical composition for promoting hair growth as a topical skin preparation. It is noted that the micelle composition also can be used as a pharmaceutical composition that is orally administered or parenterally (e.g., intravenously or intraperitoneally) administered.

### EXAMPLES

The present invention will now be described in more detail by way of examples.

### Example 1

A poly(ethylene glycol)-poly(γ-benzyl-L-glutamate) block copolymer (hereinafter referred to as “PEG-PBLG”) was used. Soybean oil was used as the fatty oil; cetylpyridinium chloride (hereinafter referred to as “CPC”), which is a cationic surfactant, was used as the charged surfactant. A known anionic peptide (hereinafter referred to as “anionic peptide A”) was used as the non-lipophilic drug. It is noted that anionic peptide A, which is a known hair growth promoter (hair growth promoting peptide), has a molecular mass of 908.94 Da and a pI of 4.95. In addition, the solubility of anionic peptide A in oil at 20° C. and standard atmospheric pressure (101.325 kPa) is in the range of 100 mg/L or less.

PEG-PBLG was prepared as follows. PEG-NH₂ (molecular mass: 10,000 Da) was dissolved in dehydrated dimethylformamide under an argon atmosphere; BLG-NCA, which is α-amino acid-N-carboxy anhydride (NCA) for polymerization of the PBLG segment, was added in an amount of 42 equivalents to PEG-NH₂, and the mixture was agitated at 40° C. for 18 hours. The resulting reaction mixture was precipitated in a mixed solvent of hexane and ethyl acetate (1:1) and then washed with the same solvent. After drying, a PEG-PBLG powder was obtained. From ¹H-NMR analysis, the degree of polymerization of the PEG segment in the PEG-PBLG was 227 and the degree of polymerization of the PBLG segment in the PEG-PBLG was 40. The structural formula of PEG-PBLG is represented by Formula (1).

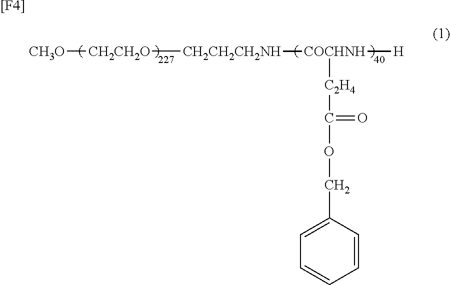

A polymeric micelle carrier composition of Example 1 was prepared as follows. 10 ml of a mixed solvent of acetone and methanol (1:1 by mass) was mixed with 300 mg of PEG-PBLG (100 parts by mass), 30 mg of CPC (10 parts by mass), and 30 mg of soybean oil (10 parts by mass). After evaporation of the solvent from the mixture, 15 mL of water was added and it was agitated; by emulsifying using an ultrahigh-pressure emulsifying apparatus (Nanovater, manufactured by Yoshida Kikai Co., Ltd.) under the conditions of 150 MPa and 5 passes, a polymeric micelle carrier composition was obtained.

A drug solution (pH 6), in which 15 mg of anionic peptide A (5 parts by mass) was dissolved in 15 ml of a 100 mM phosphate buffer, was added to the polymeric micelle carrier composition; it was agitated, and then allowed to stand still at 5° C. overnight. Thus, the polymeric micelle composition of Example 1 was prepared.

### Example 2

A carrier composition and a micelle composition were obtained in the same manner as in Example 1 except that dimethyldistearylammonium chloride (hereinafter referred to as “MSAC”), which is a cationic surfactant, was used as the charged surfactant.

### Example 3

A carrier composition and a micelle composition were obtained in the same manner as in Example 1 except that a poly(ethylene glycol)-polyleucine block copolymer (hereinafter referred to as “PEG-pLeu”) was used as the block copolymer.

PEG-pLeu was prepared in the same manner as in the PEG-PBLG of Example 1 except that Leu-NCA, which is the NCA for polymerizing the pLeu segment, was used instead of BLG-NCA, and Leu-NCA was added in an amount of 44 equivalents to PEG-NH₂. From ¹H-NMR analysis, the degree of polymerization of the pLeu segment was 40. The structural formula of PEG-pLeu is represented by Formula (2).

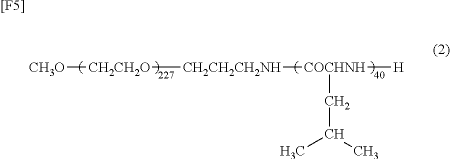

### Example 4

A carrier composition and a micelle composition were obtained in the same manner as in Example 3 except that MSAC was used as the charged surfactant.

### Example 5

A carrier composition and a micelle composition were obtained in the same manner as in Example 3 except that a known cationic peptide (hereinafter referred to as “cationic peptide B”) was used as the non-lipophilic drug, and sodium dodecyl sulfate (hereinafter referred to as “SDS”), which is an anionic surfactant, was used as the charged surfactant. Cationic peptide B has a molecular mass of 1188.38 Da and a pI of 11.8. In addition, the solubility of cationic peptide B in oil at 20° C. and standard atmospheric pressure (101.325 kPa) is in the range of 100 mg/L or less. In addition, the pH of the drug solution of Example 5 is 11.

### Example 6

A carrier composition and a micelle composition were obtained in the same manner as in Example 2 except that a poly(ethylene glycol)-poly(leucine/γ-benzyl-L-glutamate) block copolymer, composed of a PEG segment and a poly(leucine/γ-benzyl-L-glutamate) segment containing in a random manner 75 mol % leucine (Leu) units and 25 mol % γ-benzyl-L-glutamate (BLG) units, was used as the block copolymer.

Hereinafter, the mixed-type copolymer having such Leu and BLG units will be referred to as “PEG-p(Leu/BLG)”; in case the molar ratio of these units is indicated, it will be referred to as “PEG-p(Leu/BLG) (75:25).”

PEG-p(Leu/BLG) (75:25) was prepared in the same manner as in Example 1 except that Leu-NCA and BLG-NCA were used as the NCA, and the molar ratio of these NCAs was adjusted to achieve a molar ratio of Leu units and BLG units of 75:25. From ¹H-NMR analysis, the degree of polymerization of the PEG segment in PEG-p(Leu/BLG) (75:25) was 227 and the degrees of polymerization of the Leu and BLG units in the p(Leu/BLG) segment were 30 and 10, respectively.

The structural formula of PEG-p(Leu/BLG) (75:25) is represented by Formula (3). For the sake of convenience, although the Leu and BLG units are illustrated on the left and right sides, respectively in the curly brackets { } of Formulae (3), (4), and (5) described below, in fact, these units are randomly disposed.

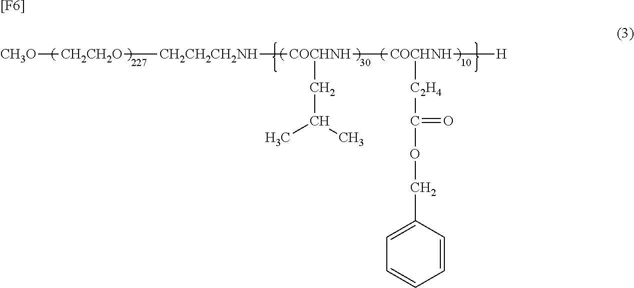

### Example 7

A carrier composition and a micelle composition were obtained in the same manner as in Example 2 except that PEG-p(Leu/BLG) (50:50) was used as the block copolymer.

PEG-p(Leu/BLG) (50:50) was prepared in the same manner as in Example 6 except that the molar ratio of the NCAs was adjusted to achieve a molar ratio of Leu units and BLG units of 50:50. The structural formula of PEG-p(Leu/BLG) (50:50) is represented by Formula (4).

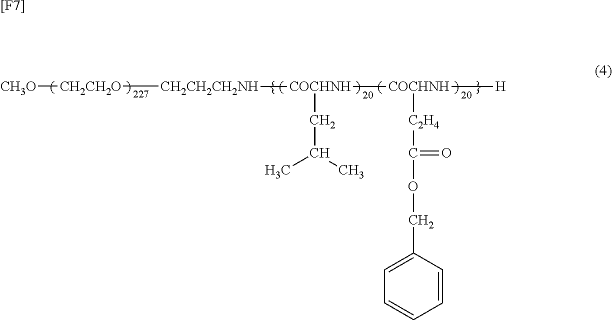

### Example 8

A carrier composition and a micelle composition were obtained in the same manner as in Example 2 except that PEG-p(Leu/BLG) (25:75) was used as the block copolymer.

PEG-p(Leu/BLG) (25:75) was prepared in the same manner as in Example 6 except that the molar ratio of the NCAs was adjusted to achieve a molar ratio of Leu units and BLG units of 25:75. The structural formula of PEG-p(Leu/BLG) (25:75) is represented by Formula (5).

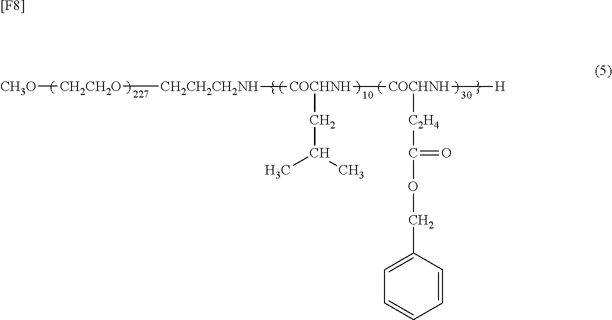

### Example 9

A carrier composition and a micelle composition were obtained in the same manner as in Example 2 except that refined sesame oil (manufactured by Summit Oil Mill Co., Ltd.) was used as the fatty oil.

### Example 10

A carrier composition and a micelle composition were obtained in the same manner as in Example 7 except that refined sesame oil (manufactured by Summit Oil Mill Co., Ltd.) was used as the fatty oil.

### Example 11

A carrier composition and a micelle composition were obtained in the same manner as in Example 4 except that refined sesame oil (manufactured by Summit Oil Mill Co., Ltd.) was used as the fatty oil.

### Comparative Example 1

A carrier composition and a micelle composition were obtained in the same manner as in Example 3 except that neither a charged surfactant nor a fatty oil was used.

### Comparative Example 2

A carrier composition and a micelle composition were obtained in the same manner as in Example 4 except that a fatty oil was not used.

### Comparative Example 3

A carrier composition and a micelle composition were obtained in the same manner as in Example 3 except that a charged surfactant was not used.

### Evaluation of the Loadability

In each of the polymeric micelle compositions of Examples 1 to 11 and Comparative Examples 1 to 3, the free amount of the non-lipophilic drug in the aqueous phase was measured by high-performance liquid chromatography (HPLC), and the drug loading percentage of each micelle composition was calculated by comparing to the amount of the drug that was added to the carrier composition. The components and drug loading percentages in each Example are shown in Table 1 below.

TABLE 1

| | | | | Non- | Drug loading |
|---|---|---|---|---|---|
| | | Charged | | lipophilic | percentage |
| | Block copolymer | surfactant | Fatty oil | drug | (mass %) |
| Example 1 | PEG-PBLG | CPC | Soybean | Anionic | 47 |
| Example 2 | | MSAC | oil | peptide A | 38 |
| Example 3 | PEG-pLeu | CPC | | | 89 |
| Example 4 | | MSAC | | | 49 |
| Example 5 | | SDS | | Cationic | 89 |
| | | | | peptide B | |
| Example 6 | PEG-p(Leu/BLG) | MSAC | | Anionic | 40 |
| | (75:25) | | | peptide A | |
| Example 7 | PEG-p(Leu/BLG) | | | | 38 |
| | (50:50) | | | | |
| Example 8 | PEG-p(Leu/BLG) | | | | 33 |
| | (25:75) | | | | |
| Example 9 | PEG-PBLG | | Refined | | 32 |
| Example 10 | PEG-p(Leu/BLG) | | sesame | | 37 |
| | (50:50) | | oil | | |
| Example 11 | PEG-pLeu | | | | 39 |
| Comparative | PEG-pLeu | — | — | Anionic | 6 |
| Example 1 | | | | peptide A | |
| Comparative | | MSAC | — | | 1 |
| Example 2 | | | | | |
| Comparative | | — | Soybean | | 9 |
| Example 3 | | | oil | | |

As shown in Table 1, the drug loading percentages in Comparative Examples 1 to 3 were less than 10%, whereas the drug loading percentages in Examples 1 to 11 were 30% or more. Thus, according to the present invention, the drug loadability of the non-lipophilic drugs in the polymeric micelle compositions could be significantly improved.

### INDUSTRIAL APPLICABILITY

The carrier composition and micelle composition of the present invention can be used in the cosmetic and pharmaceutical fields.

1. A polymeric micelle carrier composition comprising:
i) block copolymers, each having a hydrophilic polymer chain segment and a hydrophobic polymer chain segment;
ii) a charged surfactant; and
iii) a fatty oil;
wherein the block copolymers are associated in the form of a polymeric micelle having a hydrophobic region in an inner shell portion caused by hydrophobic interactions between the hydrophobic polymer chain segments of the block copolymers.

2. A polymeric micelle composition comprising:
the polymeric micelle carrier composition according to claim 1; and
a non-lipophilic drug disposed in and/or on the polymeric micelle, the non-lipophilic drug having a charge opposite to the charge of the charged surfactant.

3. The polymeric micelle composition according to claim 2,
wherein the polymeric micelle is arranged such that the hydrophobic polymer chain segments are disposed inward and the hydrophilic polymer chain segments are disposed outward,
the fatty oil is associated with the hydrophobic polymer chain segments and surrounded by the hydrophilic polymer chain segments, and
the charged surfactant is arranged around the fatty oil such that lipophilic moieties of the charged surfactant are oriented inward and hydrophilic moieties of the charged surfactant are oriented outward, the lipophilic moieties being attracted to the fatty oil.

4. The polymeric micelle composition according to claim 2, wherein the non-lipophilic drug is a charged peptide.

5. The polymeric micelle composition according to claim 2, wherein the non-lipophilic drug is a hair growth promoter.

6. The polymeric micelle composition according to claim 2, wherein the polymeric micelle composition is capable of loading the non-lipophilic drug in polymeric micelles in the range of 20 mass % or more of a total amount of the non-lipophilic drug placed into a solution with the block copolymers, the charged surfactant and the fatty oil.

7. The polymeric micelle composition according to claim 2, wherein the non-lipophilic drug is a pharmaceutical compound.

8. The polymeric micelle composition according to claim 2, wherein the non-lipophilic drug is a cosmetic compound.

9. The polymeric micelle composition according to claim 2, wherein the non-lipophilic drug is a topical skin agent.

10. A method of treatment, comprising:
orally or parenterally administering an effective amount of the polymeric micelle composition according to claim 2 to a patient in need thereof.

11. The polymeric micelle composition according to claim 4, wherein:
the polymeric micelle is arranged such that the hydrophobic polymer chain segments are disposed inward and the hydrophilic polymer chain segments are disposed outward,
the fatty oil is associated with the hydrophobic polymer chain segments and surrounded by the hydrophilic polymer chain segments, and
the charged surfactant is arranged around the fatty oil such that lipophilic moieties of the charged surfactant are oriented inward and hydrophilic moieties of the charged surfactant are oriented outward, the lipophilic moieties being attracted to the fatty oil.

12. The polymeric micelle composition according to claim 11, wherein the charged peptide is a hair growth promoter.

13. The polymeric micelle composition according to claim 12, wherein:
the block copolymers are PEG-PBLG and/or PEG-pLeu;
the charged surfactant is selected from the group consisting of cetylpyridinium chloride, dimethyldistearylammonium chloride and sodium dodecyl sulfate; and
the fatty oil is soybean oil or sesame oil.

14. The polymeric micelle composition according to claim 13, wherein:
the block copolymers are PEG-pLeu;
the charged surfactant is selected from the group consisting of cetylpyridinium chloride and sodium dodecyl sulfate; and
the fatty oil is soybean oil.

15. The polymeric micelle composition according to claim 2, wherein the hydrophilic polymer chain segment has a mass of at least 1000 Da.

16. The polymeric micelle composition according to claim 15, wherein the hydrophobic polymer chain segment has a mass of at least 1000 Da.

17. The polymeric micelle composition according to claim 16, wherein the hydrophobic polymer chain segment comprises alkyl group side chain amino acids and/or aralkyl group side chain amino acids.

18. The polymeric micelle composition according to claim 17, wherein the hydrophilic polymer chain segment contains poly(ethylene glycol).

19. The polymeric micelle composition according to claim 18, wherein the polymeric micelle composition contains:
100 parts by mass of the block copolymers,
more than 0 parts by mass and 20 parts by mass or less of the charged surfactant and
more than 0 parts by mass and 20 parts by mass or less of the fatty oil.

20. The polymeric micelle composition according to claim 19, wherein the non-lipophilic drug is a charged peptide that acts as a hair growth promoter.
